(19) United States
(12) Patent Application Publication
(10) Pub. No.: US 2017/0259027 A1
(43) Pub. Date: Sep. 14, 2017

(54) SYSTEM FOR STORING AND DISTRIBUTING AN NO/NITROGEN GASEOUS MIXTURE

(71) Applicant: Air Liquide Sante (International), Paris (FR)

(72) Inventors: Pierre DE VILLEMEUR, Houston, TX (US); Laurent LECOURT, Boulogne (FR); Catherine BESEME, Paris (FR)

(73) Assignee: Air Liquide Sante (International), Paris (FR)

(21) Appl. No.: 15/102,587

(22) PCT Filed: Nov. 28, 2014
(86) PCT No.: PCT/FR2014/053084

§ 371 Date: Jun. 8, 2016

(30) Foreign Application Priority Data
Dec. 12, 2013 (FR) 1362490

(51) Int. Cl. A61M 16/20; F17C 1/00; F17C 13/04; A61M 16/12

(52) U.S. Cl. CPC A61M 16/209; A61M 16/12; F17C 1/00; F17C 13/04; A61M 2202/0208; A61M 2202/0275; A61M 2202/0266; F17C 2201/0104; F17C 2205/0323; F17C 2205/0338; F17C 2205/0394; F17C 2221/014; F17C 2223/036; F17C 2270/025

(57) ABSTRACT

The invention relates to a system for storing and distributing a gaseous mixture formed of NO/N₂, comprising a container (6) containing an NO/nitrogen mixture equipped with an in-built pressure regulator (8) with an internal passage (22) for gas made of stainless steel and means for regulating the expansion of the gas collaborating with an expansion valve (27) and a valve seat (28) made of stainless steel. A first outlet coupling (21a) referred to as a ‘pressure’ outlet delivers the gas at low pressure and a second outlet coupling (21b) associated with a flow meter device comprising calibrated orifices and a flow rate selection member delivers the gas at several different flow rates. The invention also relates to an installation for distributing a gas containing NO to a patient comprising a ventilator (1) delivering a gas containing oxygen and to a system for storing and distributing a gaseous mixture formed of NO/N₂ according to the invention.

### CROSS REFERENCE TO RELATED APPLICATIONS

This application is a 371 of International PCT Application PCT/FR2014/053084 filed Nov. 28, 2014 which claims priority to French Patent Application No. 1362490 filed Dec. 12, 2013, the entire contents of which are incorporated herein by reference.

### BACKGROUND

The invention concerns a system for storing and distributing a gaseous mixture made up of NO/N₂, said system including a built-in pressure regulating valve or ‘PRV’ in stainless steel which is mounted on a container for conditioning the gaseous mixture of NO and nitrogen to a concentration of at least 200 ppmv (ppm in volume) and being able to go to 3500 ppmv, and at a pressure of between 100 and 500 bar.

### SUMMARY

Gaseous NO is conventionally utilized at different concentrations of between 200 and 1000 ppm in volume (hereafter referred to as ‘ppmv’), the remainder of the gaseous mixture being nitrogen, in order to treat pulmonary vasoconstrictions, notably pulmonary hypertension in patients undergoing cardiac surgery or in hypoxic newborns. Documents EP-A-786264 and EP-1516639 may be cited to this effect.

The NO/nitrogen gaseous mixture is generally conditioned at a pressure of between 100 and 200 bar absolute in a container, such as a gas cylinder, which is provided with a valve unit allowing the gas output from said container to be controlled.

In view of its very high pressure, the gas must be expanded before it is administered to the patient so as to reduce its pressure and to make it compatible to be administered by inhalation.

A gas pressure regulator which is either fixed on the valve outlet or built into the device for administering and monitoring the NO, situated downstream of the valve and being connected to said valve by means of a high-pressure type hose, is used for this purpose.

Document WO-A-99/49921 teaches an installation of this type with an NO cylinder provided with a gas distributing valve and an external gas pressure regulator which is fixed at the valve outlet.

However, these systems give rise to certain problems.

Thus, when the pressure regulator is connected to the valve by a hose, said hose harbors a risk of injury for the users in view of the high pressure which it conveys since it can be a source of improper handling.

In addition, a significant volume of gas can be contained in a hose containing high pressure gas, which potentially results in NO₂ being formed and therefore necessitates regularly purging the hose.

In the case where the pressure regulator is fixed on the outlet connection of the valve, there is the problem of overcrowding as said pressure regulator is an additional element for connection on the valve, which may for example hamper the users.

In addition, there is also a risk connected with the high pressure of the gas when the pressure regulator is installed on the cylinder. It must be possible to use the gaseous mixture containing NO very rapidly. Hence, either the pressure regulator is left on the cylinder, which gives rise to a risk of damage if the cylinder is dropped, or it is installed in retrospect, but, in this case, the installation time will delay the start of the treatment.

EP-A-2541120 also makes known a system for storing and distributing NO/N₂ said system including a container for conditioning the NO/nitrogen gaseous mixture, provided with a built-in pressure regulating valve (PRV) which allows the flow rate and the pressure of the outlet gas to be controlled.

Said type of system allows certain of the above-mentioned problems to be resolved but does not solve the one regarding compatibility between the materials making up the PRV and the stored mixtures and, in certain cases, it has been possible in practice to confirm deterioration of the passage carrying the gas and/or of the pressure-reducing elements, in particular of the valve and/or the seat of the valve.

In addition, said type of PRV does not allow the outlet flow rate to be adjusted independently of the pressure and vice versa. Similar or analogous devices are taught by documents WO-A-41856, EP-A1515080 and DE-A-19744047.

The problem to be solved, therefore, is to be able to realize a reduction in the pressure of the gaseous mixture containing NO without coming across any or part of the abovementioned problems.

The solution is a system for storing and distributing a gaseous mixture made up of NO/N₂, said system including:
a conditioning container which contains a gaseous mixture made up of NO and nitrogen, that is to say a NO/nitrogen gaseous mixture, typically the NO/N₂ gaseous mixture contains between 200 and 3500 ppm in volume of NO and the remainder is nitrogen, and
a built-in pressure regulating valve (PRV) including a valve body in metal which includes a gas inlet, at least one gas outlet and at least one internal gas passage which traverses said valve body in order to connect the gas inlet fluidically to said at least one gas outlet, said built-in pressure regulating valve being fixed on the conditioning container,
characterized in that:
at least the part of the valve body traversed by said internal gas passage is in stainless steel, and
the built-in pressure regulating valve further includes:
   means for regulating the gas expansion which cooperate with a pressure relief valve, said valve cooperating with a valve seat, the pressure relief valve and the valve seat being in stainless steel,
   a first low-pressure outlet connection which delivers low pressure gas at between 1 and 10 bar inclusive, and
   a second flow rate outlet connection which is associated with a flow meter device which includes calibrated orifices and a flow rate selecting device in order to allow the gaseous mixture containing NO/nitrogen to be delivered at several different gas flow rates, that is to say that the gas is delivered at a given flow rate which can be selected from among several different flow rates.

Thanks to the present invention, the users are no longer exposed to gaseous high pressure as the gas is expanded directly in the built-in pressure regulating valve and is therefore output at low pressure, that is to say typically between 1 and 10 bar absolute 2, for example between 2 and 7 bar absolute.

The low pressure can be fixed at a desired level thus allowing the system for delivering NO supplied by expanded gas to function well.

In addition, this also avoids the risks of improper handling which exist with the former valves and the low pressure outlet of the pressure reducing valve is able to be connected directly to a system for administering and monitoring NO.

In addition, the solution of the invention also allows a reduction in the overall space required to be obtained as it is no longer necessary to add an additional pressure reducer to the valve outlet.

Finally, the solution of the invention also allows NO to be delivered at different flow rates so as to be able to be adapted to a specific situation, like ventilation with a ventilator for administering gas or, in contrast, without a ventilator, for example in an emergency.

According to the case, the system of the invention can include one or several of the following technical characteristics:
the body is realized in part or entirely in stainless steel. The process of using stainless steel as the material constituting all or part of the passages and of the other elements in contact with the gas containing NO, in particular the valve and the valve seat, is advantageous as this allows corrosion of the elements of the pressure reducing valve as a result of the corrosive nature of the NO/gas to be avoided or minimized,
the conditioning container is cylindrical in shape,
the conditioning container includes a bottom and a neck with an outlet orifice, the built-in pressure reducing valve being fixed at the level of said outlet orifice,
the built-in pressure reducing valve is protected by a protective cover,
the concentration of NO can be identified by a given mark or color,
a failsafe system allows the risks of making the wrong connection to the built-in pressure reducing valve (PRV) to be avoided, that is to say to the first low pressure outlet connection of the PRV,
the built-in pressure reducing valve further includes means for controlling the release of the gas,
the means for regulating the expansion of the gas and/or the means for controlling the release of the gas include one or several rotary members which can be actuated manually by a user, such as one (or several) rotary wheels or a pivoting lever,
the conditioning container has an internal volume which is less than or equal to 20 liters (equivalent in water), in a preferred manner an internal volume which is less than or equal to 15 liters (equivalent in water) and more than or equal to 0.5 liters (equivalent in water),
it can comprise a device for tracking the service life of the cylinder in volume (liters) and/or in time (hours and/or minutes),
it comprises a device for tracking the service life of the cylinder designed in order to communicate information regarding the service life, notably one or more items of information regarding the service life, notably of time and/or of volume, to a system or other systems, notably a delivering apparatus, a computer or a remote server. The information regarding the service life can be transmitted to a delivering apparatus for example by infrared beam, wire, Bluetooth, GSM, GPRS or in another manner,
the first low pressure gas outlet connection of the PRV delivers gas at a pressure of between 1 and 10 bar absolute, typically of between 2 and 7 bar, in a preferred manner of between 3 and 5 bar,
the first low pressure gas outlet connection of the PRV includes failsafe means, for example a particular or analogous profile,
delivering gaseous NO at several different gas flow rates is effected via the second flow rate outlet connection,
the flow rate selecting member is a rotary knob,
the second flow rate outlet connection cooperates with the flow meter device in order to allow gaseous NO to be delivered,
the flow rate selecting member cooperates with the flow meter device in order to select one of said calibrated orifices, the diameter of the gas passage of which corresponds to the desired gas flow rate,
the gas traverses the calibrated orifice, the diameter of the gas passage of which corresponds to the desired gas flow rate, is traversed by the gaseous flow and is delivered by the second flow rate outlet connection,
the calibrated orifices, which can be selected in order to allow gaseous NO to be delivered at different given flow rates, are arranged upstream of the second outlet connection in such a manner that the gas traverses one of said calibrated orifices prior to reaching the second outlet connection through which the gas is distributed,
the calibrated orifices each have different gauges, in particular increasing diameters, corresponding to the different values of the desired flow rates,
the calibrated orifices have increasing diameters corresponding to the values of the desired flow rates of between 50 ml/min and 5 l/min inclusive, typically of between 75 ml/min and 2 l/min, in particular of between 100 ml/min and 1.5 l/min,
the calibrated orifice system can be designed in order to deliver solely a few different flow rates, for example between 2 and 6 different flow rates, so as to allow for use in degraded mode or “emergency” mode, in the event of failure of an NO delivering apparatus which is connected to the low pressure outlet of the PRV. In said “emergency” mode, the different flow rates can be fixed for example between 200 and 250 ml/min for adult patients or newborns ventilated with ventilators in HFO mode (high frequency oscillations) and between 100 and 150 ml/min for children or newborns ventilated with conventional ventilation. Indeed, such flow rate values correspond to an NO concentration of between 2 and 20 ppmv for volumes-minutes adapted to the patient in question, namely of between 2 and 30 liters for example,
the calibrated orifice system can also be designed in order to deliver more different flow rates, for example more than 10 flow rate values, so as to be able to deliver more precisely when being used without sophisticated NO delivery apparatus, for example use in an emergency vehicle (e.g., ambulance or helicopter). In this case, the flow meter part of the PRV is more complete and comprises calibrated orifices corresponding to, for example, between 10 and 15 preselected flow rates, even more than 15 flow rates, for example flow rates of between 10 ml/min and 1 l/min inclusive which correspond to flow rate values which allow NO to be administered to adult patients, children and newborns ventilated in an invasive or non-invasive manner,
the valve includes between 2 and 20 calibrated orifices having increasing diameters, typically between 2 and 15 calibrated orifices,
the calibrated orifices are developed on a rotary disk which is arranged in the gas passage upstream of the second given flow rate outlet connection,
the built-in pressure reducing valve further comprises a rupture disk and/or a thermal fuse which ensure enhanced safety in use by allowing the pressurized gas to escape into the atmosphere in the event of an accidental combustion, such as a fire, being produced in the area where the gas cylinder provided with said PRV is stored.

The invention also concerns an installation for distributing a gas containing NO to a patient, said installation including:
a ventilator which delivers a gas containing oxygen,
a system for storing and distributing a gaseous mixture made up of NO/N₂ according to the invention,
a patient circuit which is connected fluidically to the ventilator and
a device for distributing NO which allows the quantity of NO/N originating from the storage and distribution system and released into the patient circuit to be controlled.

Furthermore, the invention also concerns a method for distributing a mixture containing NO/N₂, which method uses a system for storing and distributing a gaseous mixture made up of NO/N₂ according to the invention or an installation for distributing a gas containing NO to a patient according to the invention.

### BRIEF DESCRIPTION OF THE DRAWINGS

The present invention will now be better understood thanks to the description given below with reference to the accompanying figures, in which:

FIG. 1 is an embodiment of an installation for distributing NO which is supplied by a system for conditioning gas according to the invention and

FIG. 2 is a general schematic diagram of the operation of a built-in pressure reducing valve (PRV) of a system for storing and distributing gas which complies with the present invention.

### DESCRIPTION OF PREFERRED EMBODIMENTS

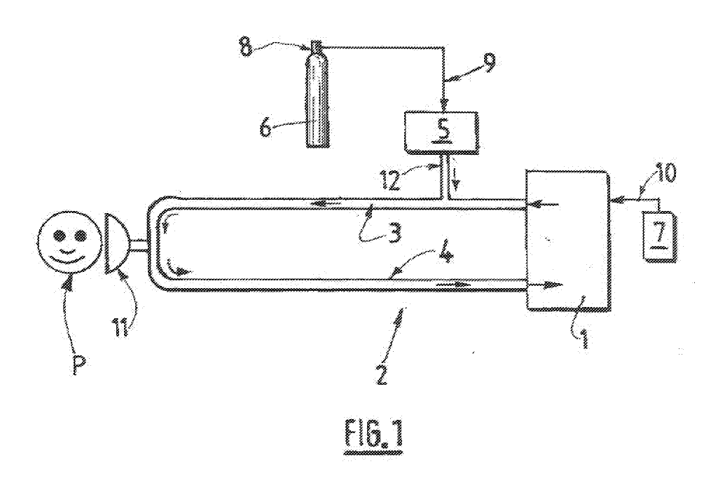

The storing and distributing system according to the invention can be used in order to supply an installation for distributing NO to patients suffering from pulmonary vasoconstrictions, for example via the NO distributing installation, an embodiment of which is shown in FIG. 1.

Said installation includes a ventilator 1 with a respiratory circuit or patient circuit 2 with two branches, that is to say one inspiratory branch 3 and one expiratory branch 4. The inspiratory branch 3 is designed to route the respiratory gas from the ventilator to the patient P, whilst the expiratory branch 4 is designed to route the gas exhaled by the patient P to the ventilator 1.

In terms of the patient P, the administering of the gas is effected by means of a patient interface 11, for example a respiratory mask, a tracheal cannula or nasal cannula.

The ventilator 1 is supplied, via a connecting line 10, with a gas containing oxygen, for example air (O₂ content 21% by volume) or the oxygen is delivered from an oxygen source 7, such as an oxygen cylinder or a pipe carrying oxygen originating from an oxygen producing unit, such as a modulated pressure unit (PSA), or an oxygen storage unit, such as a buffer tank or storage tank.

The oxygen-rich gas is delivered into the inspiratory branch 3 of the patient circuit 2 by the ventilator 1.

Moreover, a device 5 for distributing NO is connected fluidically to said inspiratory branch 3 of the patient circuit 2 in order to deliver there, via a supply line 12, an NO/N mixture, for example 200, 400, 800 or 1500 ppmv NO and the remainder being nitrogen.

The device 5 for distributing NO is itself supplied with NO/N mixture, via a gas supply line 9, by a NO/N₂ container which is part of a system for storing and distributing according to the invention.

The NO/N₂ container 6 is a gas cylinder, for example produced in composite fibers, in aluminum or in an aluminum alloy containing 0.5, 2, 5, 10, 11 or 20 liters (equivalent in water), provided with a built-in pressure reducing valve 8, in a preferred manner protected against impacts by means of a protective cover.

The built-in pressure reducing valve 8, called a “PRV”, allows the outlet of the gas from the container 6 and its outlet pressure to be controlled, notably by means of a pressure relief valve 27 and a valve seat 28.

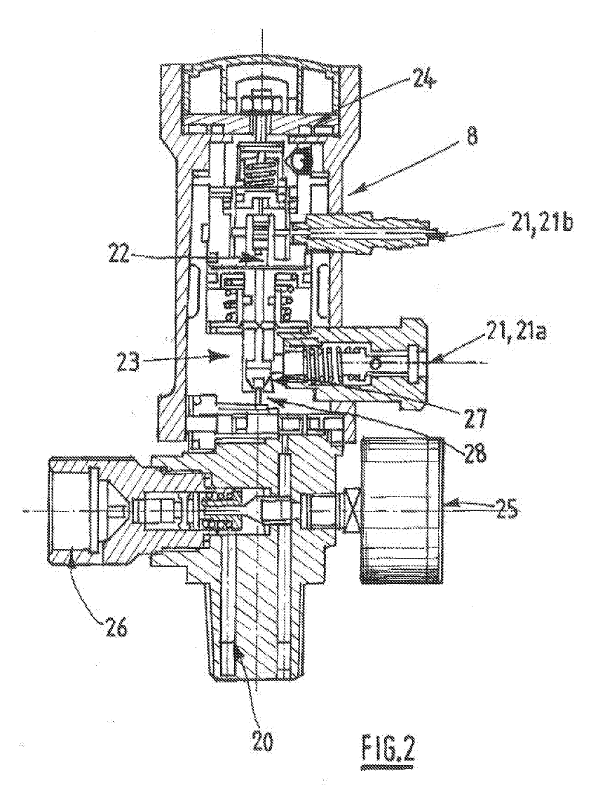

Said PRV 8 is made up by a body which is traversed by one or several gas passages 22 which connect a gas inlet 20, which is situated at the fixing end of the PRV on the neck of the cylinder, to one or several outlet connections 21 through which the gas leaves the PRV again, after expansion, as detailed below with reference to FIG. 2.

FIG. 2 shows an embodiment of a built-in pressure reducing valve or “PRV” which is produced in stainless steel and is designed to be mounted on a container for conditioning the gaseous mixture of NO and nitrogen, in particular by means of screw-connection on the neck of a gas cylinder.

Said PRV 8 includes a valve body 23 (seen in section) which includes an internal gas passage 22 which connects, on the one hand, a gas inlet 20, by means of which the NO/nitrogen mixture is extracted from the body of the gas cylinder 6, on which said PRV 8 is mounted, and, on the other hand, several gas outlets 21, that is to say gas outlet connections, by means of which the NO/nitrogen mixture leaves the PRV 8, before being conveyed to the patient P, as explained above with reference to FIG. 1.

According to the invention, the PRV includes two gas outlet connections 21, 21a, 21b which include:
a first so-called “pressurized” gas outlet connection 21a which delivers low pressure gas of typically between 1 and 10 bar inclusive, to which gas delivering apparatuses are connected. The first gas outlet connection 21a is supplied by gas which originates from the high pressure gas source and has been expanded by the expanding means of the PRV, in particular the valve and the valve seat.
and a second given flow rate outlet connection 21b which delivers gas flow rates which are selected corresponding to predetermined volumes so as to supply different gas flow rates, for example between 5 and 20 different flow rates, typically between 10 and 15 different flow rates, which are chosen by the user in terms of the patient in question, for example an adult or child. The second outlet connection therefore allows for gaseous NO to be delivered at different gas flow rates which can be selected by means of several calibrated orifices with increasing diameters. The calibrated orifices with different diameters form part of a flow meter type device or system. In a preferred manner, the calibrated orifices are developed on a rotary disk which is arranged in the gas passage upstream of the second given flow rate outlet connection 21b. The second outlet connection 21b therefore allows gas to be delivered at different flow rate values of between 50 ml/min and 5 l/min inclusive, typically of between 75 ml/min and 2 l/min, in particular of between 100 ml/min and 1.5 l/min.

One or several control members 24, such as a rotary member which is able to be handled by an operator, allow the release of the gas to be controlled and the desired flow rate to be selected. This is therefore a gaseous flow rate selector, for example a knob or a rotary wheel.

In particular, a flow rate control member 24 acts on the rotary disk so as to be able to ensure that a selection of the desired flow rate value is delivered by the second outlet connection 21b.

So as to minimize the corrosion of the elements of the PRV 8 in view of the corrosive nature of the NO/nitrogen gaseous mixture, the body 23 of the PRV 8 is realized entirely or in part, but preferentially entirely, in stainless steel.

Indeed, stainless steel is used as the material constituting the passages and other elements in contact with the gas containing NO. Thus, the gas passage or passages are in particular drilled through stainless steel parts of the body 3 of the PRV 8. In the same way, the pressure relief valve 27 and/or the valve seat 28, and/or other elements are also produced in stainless steel.

The PRV 8 further comprises a manometer 25 and a filling connection 26 which allows the cylinder 6 to be filled with gas when it is empty, that is to say when all the gas has been consumed. The PRV 8 comprises or can comprise, moreover, other classic components, such as springs, gaskets, etc.

Furthermore, the device 5 for distributing NO notably allows the quantity of NO/N released into the inspiratory branch 3 to be controlled, as well as the mode of releasing said mixture, that is to say in a continuous manner or in a pulsed manner, for example solely during the inspiratory phases of the patient P. The NO/N mixture is therefore diluted in the inspiratory branch 3 with the O₂-rich mixture distributed by the ventilator 1. The diluting is a function of the content of the initial NO/N mixture but also of the gas concentration to be administered to the patient.

It will be understood that many additional changes in the details, materials, steps and arrangement of parts, which have been herein described in order to explain the nature of the invention, may be made by those skilled in the art within the principle and scope of the invention as expressed in the appended claims. Thus, the present invention is not intended to be limited to the specific embodiments in the examples given above.

(For Allen) While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as fall within the spirit and broad scope of the appended claims. The present invention may suitably comprise, consist or consist essentially of the elements disclosed and may be practiced in the absence of an element not disclosed. Furthermore, if there is language referring to order, such as first and second, it should be understood in an exemplary sense and not in a limiting sense. For example, it can be recognized by those skilled in the art that certain steps can be combined into a single step.

The singular forms “a”, “an” and “the” include plural referents, unless the context clearly dictates otherwise.

“Comprising” in a claim is an open transitional term which means the subsequently identified claim elements are a nonexclusive listing (i.e., anything else may be additionally included and remain within the scope of “comprising”). “Comprising” as used herein may be replaced by the more limited transitional terms “consisting essentially of” and “consisting of” unless otherwise indicated herein.

“Providing” in a claim is defined to mean furnishing, supplying, making available, or preparing something. The step may be performed by any actor in the absence of express language in the claim to the contrary.

Optional or optionally means that the subsequently described event or circumstances may or may not occur. The description includes instances where the event or circumstance occurs and instances where it does not occur.

Ranges may be expressed herein as from about one particular value, and/or to about another particular value. When such a range is expressed, it is to be understood that another embodiment is from the one particular value and/or to the other particular value, along with all combinations within said range.

All references identified herein are each hereby incorporated by reference into this application in their entireties, as well as for the specific information for which each is cited.

1. A system for storing and distributing a gaseous mixture made up of NO/N₂, said system including:
a container (6) for conditioning gas which contains a gaseous mixture made up of NO and nitrogen, and
a built-in pressure regulating valve (8) including a valve body (D) in metal which includes a gas inlet (20), at least one gas outlet (21; 21a, 21b) and at least one internal gas passage (22) which traverses said valve body (23) in order to connect the gas inlet (20) fluidically to said at least one gas outlet (21; 21a, 21b), said built-in pressure regulating valve (8) being fixed on the conditioning container (6),
wherein:
at least the part of the valve body (23) traversed by said internal gas passage (22) is in stainless steel, and
the built-in pressure regulating valve (8) further includes:
a subsystem for regulating the gas expansion which cooperate with a pressure relief valve (27), said valve (27) cooperating with a valve seat (28), the pressure relief valve (27) and the valve seat (28) being in stainless steel,
a first pressurized outlet connection (21a) which is configured for and adapted to delivers a low pressure gas between 1 and 10 bar inclusive, and
a second flow rate outlet connection (21b) which is associated with a flow meter device which includes calibrated orifices and a flow rate selecting device configured for and adapted to allow the gaseous mixture containing NO/nitrogen to be delivered at several different gas flow rates.

2. The system of claim 1, wherein the valve body (23) is entirely in stainless steel.

3. The system of claim 1, wherein the conditioning container (6) is cylindrical in shape and includes a bottom and a neck with an outlet orifice, the conditioning container (6) being fixed at the level of said outlet orifice, and the built-in pressure regulating valve (8) is protected by a protective cover.

4. The system of claim 1, wherein the valve (8) includes between 2 and 20 calibrated orifices each with different diameters.

5. The system of claim 1, wherein the built-in pressure regulating valve (8) further includes means for controlling the release of the gas.

6. The system of claim 1, wherein the subsystem for regulating the expansion of the gas and/or the flow rate selection device include one or more rotational members (24) which can be actuated manually by a user.

7. The system of claim 1, wherein the calibrated orifices are arranged upstream of the second outlet connection (21b) such that the gas traverses one of said calibrated orifices before reaching the second outlet connection (21b).

8. The system of claim 1, wherein the calibrated orifices have increasing diameters corresponding to different flow rates.

9. The system of claim 1, wherein the conditioning container (6) has an internal volume which is less than or equal to 20 liters (equivalent in water).

10. The system of claim 1, wherein the system further comprises a device configured for and adapted to track the operating life of the cylinder in volume remaining and/or in time remaining.

11. The system of claim 1, wherein the calibrated orifices have increasing diameters corresponding to flow rates of between 50 ml/min and 5 l/min inclusive.

12. The system of claim 1, wherein the calibrated orifices are provided by a rotary disk which is arranged in the gas passage upstream of the second outlet connection (21b).

13. The system of claim 1, wherein the outlet connection for the low pressure gas includes a failsafe device.

14. An installation for distributing a gas containing NO to a patient, said installation including:
a ventilator (1) which delivers a gas containing oxygen,
a system for storing and distributing a gaseous mixture made up of NO/N2 as claimed in claim 1,
a patent circuit (2) which is connected fluidically to the ventilator (1), and
a device (5) for distributing NO which is configured for and adapted to control the quantity of NO/N2 originating from the storage and distribution system and released into the patient circuit (2).

15. A method for distributing a mixture containing NO/N2, which method uses a system for storing and distributing a gaseous mixture made up of NO/N2 as claimed in claim 1.
